(19) United States
(12) Patent Application Publication
(10) Pub. No.: US 2019/0125239 A1
(43) Pub. Date: May 2, 2019

(54) METHOD AND APPARATUS FOR PREDICTING POSTTRAUMATIC BEHAVIOR PROBLEM

(71) Applicant: Ewha University - Industry Collaboration Foundation, Seoul (KR)

(72) Inventors: In Kyoon LYOO, Seoul (KR); Han Byul CHO, Gyeonggi-do (KR); Ga Hae HONG, Gyeonggi-do (KR)

(73) Assignee: Ewha University - Industry Collaboration Foundation, Seoul (KR)

(21) Appl. No.: 15/882,254

(22) Filed: Jan. 29, 2018

(30) Foreign Application Priority Data
Nov. 2, 2017 (KR) 10-2017-0145501

(51) Int. Cl. A61B 5/00; G16H 50/30; G01N 33/68; G01N 33/74; G16H 50/20

(52) U.S. Cl. CPC A61B 5/4088; G16H 50/30; G01N 33/6896; G01N 2800/301; G16H 50/20; G01N 2800/2814; G01N 33/74

(57) ABSTRACT

Provided is a method and apparatus for predicting a posttraumatic behavior problem that may predict a posttraumatic violent behavior problem of an individual, in detail, that may determine a biological phenotype of an individual experiencing a traumatic event within a predetermined period after the individual is exposed to the traumatic event, predict a violent symptom presentation probability of the individual based on the biological phenotype of the individual, and suggest an objective basis for preventive intervention in a development of posttraumatic stress disorder (PTSD) of the individual based on a prediction result.

### TECHNICAL FIELD

Embodiments relate to a method and apparatus for predicting a posttraumatic behavior problem, and more particularly, to a method of predicting a behavior problem that may occur in the future from neurophysiological phenomena and symptoms presented by an individual immediately after exposure to a traumatic event.

### BACKGROUND ART

Posttraumatic stress disorder (PTSD) includes a maladjustive response of an individual to an environmental stress event provided externally in life, a mental response of a patient experiencing a traumatic event, and a neurophysiological response related to stress. It is known that a brain process is based on such responses. PTSD may develop immediately after the stress event, or may not develop until weeks, months, or years after the stress event. That is, PTSD is a disorder of feeling excessive anxiety after watching or directly experiencing an accident, violence, or a disaster. Different PTSD symptoms are presented based on the frequency and strength of watching or experiencing such trauma, and a neurophysiological characteristic of an individual.

The PTSD symptoms include re-experiencing, avoidance, and hyperarousal. In detail, re-experiencing is a symptom of frequently having sudden memories of a traumatic scene, and experiencing the same feeling at that time again. Avoidance is a symptom, opposite to re-experiencing, of avoiding mentioning a traumatic event. Hyperarousal is a symptom of being oversensitive to a little sound or motion due to hyper-sensitive nerves.

PTSD causes physical distress such as headache, stomachache or muscular pain, or improves mental symptoms such as depression, a personality disorder, anxiety disorder or schizophrenia. PTSD patients may be addicted to alcohol or drugs to relieve pain, or show extreme behaviors. In particular, PTSD patients showing behavioral symptoms remarkably are at a risk of violence or self-harm, which is also a serious social issue. Thus, it is mightily significant to predict a behavior problem to be presented after exposure to trauma.

Cognitive behavioral therapy (CBT) is a representative treatment that may minimize PTSD-related problems. CBT makes a PTSD patient confront memories of a traumatic event that the patient consistently suppresses and avoids, thereby treating distorted cognition about the patient, others and the world. CBT is performed in combination with drug treatment and psychotherapy.

However, in reality, a small portion of patients take actions against a development of PTSD or receive treatment before the development of PTSD since a few realize problems of PTSD. In addition, it takes time to treat PTSD patients who are overly nervous and show drastic emotional changes in response to slight actions or speeches of others.

Further, currently there is no method to detect a development of PTSD before patients present and express PTSD symptoms. However, even before the presentation of such symptoms, a neurophysiological response, a hematological response, and a neuroimaging characteristic change slightly. Thus, by analyzing the neurophysiological response, the hematological response, and the neuroimaging characteristic (hereinafter, posttraumatic physical response information) preceding the presentation of symptoms, the presentation of PTSD symptoms may be predicted. In particular, behavioral symptoms such as violence and self-ham are serious issues and greatly affect the society, and thus suitable treatment is to be applied by predicting a symptom presentation risk before the presentation of symptoms. However, each time of the posttraumatic physical response information has a little effect size to predict the presentation of PTSD symptoms, and thus an approach to efficiently integrate and analyze the items of the posttraumatic physical response information is essential.

Accordingly, there is needed a method of verifying posttraumatic physical response information of a patient that may change before a development of PTSD after experiencing a traumatic event, and predicting a PTSD-related problem presentation probability in advance by integrally analyzing the posttraumatic physical response information.

### DISCLOSURE OF INVENTION

### Technical Goals

An aspect provides a method of predicting a behavior problem that may verify whether an individual is classified as a biological phenotype based on posttraumatic physical response information of the individual obtained within first three months after the individual is exposed to a traumatic event, extract a violent behavior risk group based on the classified biological phenotype, and predict a violent symptom presentation probability of the individual through the extracted violent behavior risk group.

Another aspect provides a method of predicting a behavior problem that may obtain posttraumatic physical response information of an individual within three months immediately after the individual is exposed to trauma, known as a prime time for preventive intervention in a development of posttraumatic stress disorder (PTSD), predict a violent behavior risk group with a high violent behavioral symptom presentation probability of the individual, and apply preventive intervention suitable for the predicted violent behavior risk group to the individual.

### Technical Solutions

According to an aspect, there is provided a method of predicting a posttraumatic behavior problem, the method including classifying an individual based on a type of a traumatic event to which the individual is exposed, obtaining, from the individual, primary posttraumatic physical information of the individual within a predetermined period after the exposure to the traumatic event, determining a violent behavior risk group based on a biological phenotype of the individual by analyzing the primary posttraumatic physical information, obtaining secondary posttraumatic physical information of the individual to determine a specificity of the violent behavior risk group, and predicting a violent symptom presentation probability of the individual based on the primary posttraumatic physical information and the secondary posttraumatic physical information.

The type of the traumatic event may be classified as physical violence, sexual violence, accidents/disasters, or others based on a characteristic of the traumatic event.

The primary posttraumatic physical information may include a neurophysiological element related to autonomic nerve modulation, and a blood substance element related to oxidative stress, neuroregeneration and a hypothalamic-pituitary-adrenal axis (HPA) of the individual.

The determining may include classifying the individual as one of an emotional phenotype, a behavioral phenotype, and a cognitive phenotype by applying a cluster analysis based on the primary posttraumatic physical information, and determining a biological phenotype corresponding to the classified phenotype to be the violent behavior risk group of the individual.

The biological phenotype may have a characteristic of a biased phenotype of a predetermined symptom expressed by the individual for a short period of time or a long period of time as a mental maladjustment symptom after the individual is exposed to the traumatic event.

The obtaining of the secondary posttraumatic physical information may include obtaining, as the secondary posttraumatic physical information, neuroimaging information about a brain reward circuit related to a violent behavior in brain tissue.

The predicting may include predicting the violent symptom presentation probability of the individual in view of a correlation between elements based on the primary posttraumatic physical information and the secondary posttraumatic physical information.

According to another aspect, there is also provided an apparatus for predicting a posttraumatic behavior problem, the apparatus including a processor configured to classify an individual based on a type of a traumatic event to which the individual is exposed, obtain, from the individual, primary posttraumatic physical information of the individual within a predetermined period after the exposure to the traumatic event, determine a violent behavior risk group based on a biological phenotype of the individual by analyzing the primary posttraumatic physical information, obtain secondary posttraumatic physical information of the individual to determine a specificity of the violent behavior risk group, and predict a violent symptom presentation probability of the individual based on the primary posttraumatic physical information and the secondary posttraumatic physical information.

### BRIEF DESCRIPTION OF DRAWINGS

FIG. 1 illustrates an apparatus for predicting a posttraumatic violent behavior problem according to an embodiment.

FIGS. 2a through 2c illustrate posttraumatic physical response information of an individual according to an embodiment.

FIG. 3 illustrates an operation of predicting a presentation of a violent symptom according to an embodiment.

FIGS. 4a and 4b are graphs illustrating characteristics of biological phenotypes according to an embodiment.

FIG. 5 is a flowchart illustrating a method of predicting a posttraumatic behavior problem according to an embodiment.

FIG. 6 is a block diagram illustrating an example of predicting a violent behavior problem of an individual according to an embodiment.

### BEST MODE FOR CARRYING OUT THE INVENTION

Hereinafter, reference will now be made in detail to embodiments with reference to the accompanying drawings.

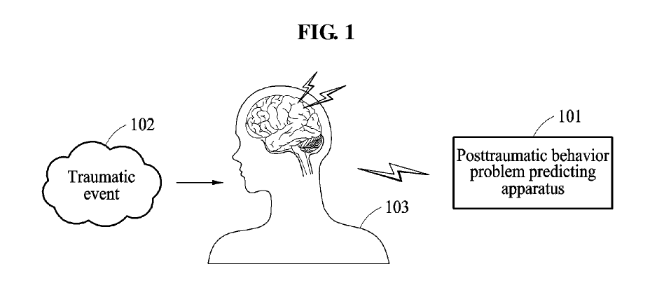

FIG. 1 illustrates an apparatus for predicting a posttraumatic violent behavior problem according to an embodiment.

Referring to FIG. 1, a posttraumatic behavior problem predicting apparatus 101 may predict a violent symptom presentation probability based on neurophysiological phenomenon information, hematological change information, and neuroimaging information of an individual 103 exposed to a traumatic event 102. In detail, the traumatic event 102 is a distressing event that the individual 103 may experience in life, and includes various incidents such as traffic accidents, murder incidents, and natural disasters that impair general adaptability. A type of the traumatic event 102 may be classified as physical violence, sexual violence, accidents/disasters, or others based on a characteristic of an event or incident applied to the individual 103. The individual 103 may directly or indirectly experience the traumatic event 102 unwillingly, and present mental symptoms of a maladjustment response after experiencing the traumatic event 102.

\textcircled{1} Physical violence is a traumatic event that applies physical damage to a body of an individual or brings a financial loss, and may correspond to direct physical violence. For example, physical violence may include bullying and gang assaults.

\textcircled{2} Sexual violence is a traumatic event in which an individual is sexually assaulted against will of the individual, and may correspond to all sexual acts and assaults by mental or psychological pressure. For example, sexual violence may include sexual harassment, indecent assaults, and rape.

\textcircled{3} Accidents/disasters are one-session traumatic events, and may correspond to man-made incidents that occur unexpectedly or abnormal natural phenomena. For example, accidents/disasters may include earthquake, typhoon, flood, drought, tsunami, fire, and epidemic of a disease.

\textcircled{4} Others may correspond to traumatic events that do not belong to physical violence, sexual violence, and accidents/disasters described above. For example, others may include death of a parent, spouse or child, and severe stress.

A neurophysiological phenomenon, a hematological change, and a neuroimaging change may be observed immediately or at a relatively early stage after the exposure to the traumatic event 102 before posttraumatic stress disorder (PTSD) symptoms are presented. The PTSD symptoms may be presented within a short period of time, or may be hidden for a long time and presented in an unexpected situation based on a type of the traumatic event 102, an exposure count, interpersonal involvement, and a neurophysiological characteristic of an individual.

In view of the foregoing, the posttraumatic behavior problem predicting apparatus 101 may obtain posttraumatic physical response information of the individual 103 within three months immediately after the exposure to the traumatic event 102. In particular, preventive intervention in a violent behavior may be effective within three months after exposure to trauma. Thus, it is important to predict a violent behavior within three months after the exposure to trauma. That is, a period of three months after a development of PTSD is known as a prime time for preventive intervention in PTSD symptoms. Although the symptoms are not presented within three months immediately after the trauma, it is extremely important to predict a violent behavior risk group with a high violent behavior symptom presentation probability and apply suitable preventive intervention.

The traumatic event 102 may be a personal issue of the individual 103 exposed to the traumatic event 102, and also be a social issue. Thus, the posttraumatic behavior problem predicting apparatus 101 may detect a physical change in the individual 103 within a short period of time after the individual 103 experiences the traumatic event 102.

The posttraumatic physical response information may be obtained by being classified into primary posttraumatic physical information and secondary posttraumatic physical information to determine a specificity related to violence of an individual, a time, or a point in time at which information is obtained from the individual. For example, the primary posttraumatic physical information of the individual may be obtained within a predetermined period after exposure to a traumatic event. In addition, the secondary posttraumatic physical information of the individual may be obtained to determine a specificity of a violent behavior risk group of the individual classified based on the primary posttraumatic physical information. That is, as information (predictors) to be used to predict and determine a violent behavior of the individual in advance, the posttraumatic physical response information including the primary posttraumatic physical information and the secondary posttraumatic physical information may be obtained.

When obtaining the primary posttraumatic physical information, different types of physical response information may be obtained to determine a specific posttraumatic response presented for each type of the traumatic event that the individual experiences. A different event may be imprinted on a brain of the individual based on a type of the traumatic event that the individual experiences. Thus, a different posttraumatic response may be presented by the individual. That is, an individual having experienced a “traffic accident” and an individual having experienced the “Iraq War” may have similar direct/indirect experiences of death. However, the two individuals may present different posttraumatic responses due to different causes of the experiences of death, and thus different types of information may need to be obtained to determine the different posttraumatic responses.

Accordingly, a different type of posttraumatic physical response information to be used to predict a biological phenotype may be obtained based on a type of a traumatic event to which an individual is exposed, and thus a different prediction model may be applied based on the type of the traumatic event.

To determine PTSD symptoms presented by the individual 103 more objectively, a neurophysiological element and a blood substance element may be obtained as the primary posttraumatic physical information. The posttraumatic behavior problem predicting apparatus 101 may obtain information about elements that may physically change in response to a traumatic event after exposure to the traumatic event. The posttraumatic behavior problem predicting apparatus 101 may determine a biological phenotype based on a characteristic of a biological phenotype group of the individual by applying a cluster analysis based on the primary posttraumatic physical information. In detail, specific posttraumatic physical response information may be obtained based on each type of the traumatic event. A behavioral trauma presentation level and a type of posttraumatic physical response information to be used to predict the same may be different based on the type of the traumatic event, and thus a posttraumatic behavior problem predicting apparatus corresponding to each type of the traumatic event may be established. The primary posttraumatic physical information corresponding to each type of the traumatic event may form a cluster of groups having a high index of correlation. Herein, three clusters may be defined and classified based on posttraumatic responses. Based on the cluster analysis, each individual may be classified as one of an emotional phenotype, a behavioral phenotype, and a cognitive phenotype.

That is, the biological phenotype of the individual may be classified through the cluster analysis from the primary posttraumatic physical information based on the type of the traumatic event. In an example, a presentation of a posttraumatic symptom of the individual may be classified as the cognitive phenotype based on primary posttraumatic physical information of the individual experiencing physical violence. The presentation of the posttraumatic symptom of the individual may be classified as the emotional phenotype based on primary posttraumatic physical information of the individual experiencing accidents/disasters. Since a different biological phenotype is expressed based on the type of the traumatic event and how the individual accepts the traumatic event, a violent behavior risk group corresponding to the type of the traumatic event may be adaptively determined and classified based on a condition of each individual, rather than being preset.

The posttraumatic behavior problem predicting apparatus 101 may additionally obtain the secondary posttraumatic physical information including neuroimaging information specific to a violent behavior, after the primary posttraumatic physical information is obtained. The posttraumatic behavior problem predicting apparatus 101 may utilize the primary posttraumatic physical information and the secondary posttraumatic physical information to predict a violent behavior. A nonlinear prediction equation to be used to predict a violent behavior may be established in advance through a sample group.

That is, to establish the nonlinear prediction equation to be applied to the posttraumatic behavior problem predicting apparatus 101, a sample group may be formed based on each type of the traumatic event, and posttraumatic physical response information of individuals may be obtained. The posttraumatic behavior problem predicting apparatus 101 may analyze an odds ratio to predict a violent behavior problem based on the posttraumatic physical response information, and extract factors with relatively high odds ratios from the posttraumatic physical response information. Then, the posttraumatic behavior problem predicting apparatus 101 may secondarily extract factors with a lowest correlation therebetween from the extracted factors with the relatively high odds ratios. An operation of extracting factors with a lowest correlation may be a process of selecting a minimum number of factors by excluding factors having duplicate effects, in a nonlinear violent behavior prediction model. The secondarily extracted factors may be used to establish the nonlinear equation to predict a behavior problem for a sample group corresponding to the type of the traumatic event. In this example, a nonlinear equation exhibiting a highest prediction rate using a minimum number of factors may be selected.

Here, the nonlinear violent behavior prediction model may be represented using a series of process of extracting a minimum number of specific posttraumatic response information through machine learning using posttraumatic physical response information (predictors) and trauma group information (outcome or trauma characteristics) of a sample group selected in advance based on the type of the traumatic event, and establishing a nonlinear equation utilizing the extracted information.

Through the above process, a nonlinear equation and factors of the posttraumatic physical response information finally selected from the sample group based on each type of the traumatic event may be selected finally. Based on the nonlinear equation and the factors of the posttraumatic physical response information, posttraumatic response information may be obtained by selecting factors of the posttraumatic physical response information within one month after exposure to trauma, and a behavior problem prediction rate may be extracted by applying the nonlinear equation.

The posttraumatic behavior problem predicting apparatus 101 may objectify a negative response of the individual to the traumatic event by complexly examining each element. The posttraumatic behavior problem predicting apparatus 101 may classify the individual as one of the emotional phenotype, the behavioral phenotype, and the cognitive phenotype by applying the cluster analysis based on the primary posttraumatic physical information. The posttraumatic behavior problem predicting apparatus 101 may determine a biological phenotype corresponding to the classified phenotype to be the violent behavior risk group of the individual. Here, the biological phenotype may have a characteristic of a biased phenotype of a predetermined symptom expressed by the individual for a short period of time or a long period of time as a mental maladjustment symptom after the individual is exposed to the traumatic event. In an example, the characteristic of the biological phenotype may indicate each posttraumatic symptom that may be presented by the individual, for example, depression, impulsivity, anger, alcohol use, attention, or emotion recognition.

The posttraumatic behavior problem predicting apparatus 101 may predict a violent symptom presentation probability, for individuals belonging to a risk group belonging to the violent behavior risk group, that is, one of the emotional phenotype, the behavioral phenotype, and the cognitive phenotype. That is, the posttraumatic behavior problem predicting apparatus 101 may predict an individual with a high probability of presenting a behavior problem such as violence or self-harm after exposure to a traumatic event based on a biological phenotype of the individual. For example, the posttraumatic behavior problem predicting apparatus 101 may discover, at an early stage, a violent behavior risk group (behavioral type) with a high probability of presenting violence within one to three months after the exposure to the traumatic event. The violence presented after the exposure to the traumatic event may be determined based on a level of addition (dependence) to alcohol and illegal drugs, a frequency of self-harm and suicide, a predetermined or higher frequency of impulsive behavior, a frequency of anger-out behavior, or a frequency of harming others and aggressive behavior.

Here, the posttraumatic behavior problem predicting apparatus 101 may determine the violent behavior risk group of the individual using indicators of a neurophysiological element, a blood substance element, and a neurological element (neuroimaging) as the posttraumatic physical response information indicating a physical change in the individual, thereby efficiently and accurately extracting a high violent behavior risk group with a high probability of presenting violence after trauma.

Ultimately, the posttraumatic behavior problem predicting apparatus may predict a behavioral response to be presented by an individual in advance based on a principle of conditioning by exposure to a traumatic event, and provide criteria for receiving suitable treatment with respect to the behavioral response, thereby suggesting a basis for preventive prediction and intervention in posttraumatic violence. The behavioral response may refer to a symptom of more intensively presenting a behavior to reduce anxiety caused by severe pain and stress as an anxiety response to a conditioned stimulus is conditionally formed by stimulation of an individual by a traumatic event or a cue related to the traumatic event.

That is, the posttraumatic behavior problem predicting apparatus 101 may predict violence of the individual 103 to be triggered by negative thoughts or emotions of the individual 103 exposed to the traumatic event 102, and suggest a basis for inducing treatment of the individual 103 to change the violence to an effective alternative behavior.

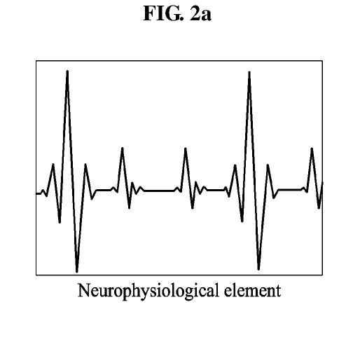

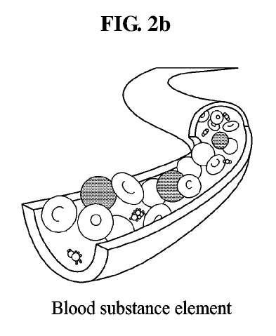

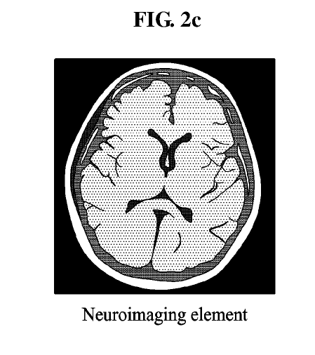

FIGS. 2a through 2c illustrate posttraumatic physical response information of an individual according to an embodiment.

Referring to FIGS. 2a through 2c, a posttraumatic behavior problem predicting apparatus may obtain posttraumatic physical response information to predict a high violent behavior risk group with a high probability of presenting violence, for an individual exposed to a traumatic event. The posttraumatic behavior problem predicting apparatus may obtain the posttraumatic physical response information in three domains to determine a physical change in the individual in response to a mental symptom.

The posttraumatic behavior problem predicting apparatus may obtain a neurophysiological element and a blood substance element to complexly examine elements that may be associated with a response to a traumatic event. In detail, the posttraumatic behavior problem predicting apparatus may determine a posttraumatic response to be presented specifically based on a type of the traumatic event from the neurophysiological element and the blood substance element. In detail, primary posttraumatic physical information may be obtained from all individuals exposed to traumatic events. In this example, all the individuals correspond to a group of individuals experiencing different traumatic events, and may present different posttraumatic responses based on types of the traumatic events. In an example, an individual experiencing a traumatic event of which a type is classified as physical violence may have a high concentration of a substance that represents an inflammation-immune system in the blood in response to an event of substantially applying physical harm. An individual experiencing a traumatic event of which a type is classified as sexual violence may have a high concentration of a substance that represents a female sex hormone. Ultimately, the posttraumatic behavior problem predicting apparatus may obtain a different type of primary posttraumatic physical information based on a type of a traumatic event in response to a posttraumatic response to be presented as a maladjustment symptom of an individual exposed to the traumatic event.

The posttraumatic behavior problem predicting apparatus may obtain secondary posttraumatic physical information including neuroimaging information of the individual as a neurologic element. In this example, the neurophysiological element and the blood substance element that may be relatively easy to collect may be defined as primary basic posttraumatic physical information, and a neuroimaging element that may be relatively difficult to collect but have a relatively high specificity to predict a violent behavior may be defined as secondary intensive posttraumatic physical response information.

Referring to FIG. 2a, the posttraumatic behavior problem predicting apparatus may obtain the neurophysiological element as an element related to autonomic nerve modulation of the individual. In detail, the posttraumatic behavior problem predicting apparatus may obtain the neurophysiological element to determine a condition with respect to physical function modulation as a maladjustment symptom of the individual exposed to the traumatic event. The posttraumatic behavior problem predicting apparatus may measure, as the neurophysiological element, a heart rate, a heart rate variability (HRV), or a skin conductance (SC) of the individual. In particular, the heart rate and the HRV may be classified as the primary posttraumatic physical information, and the SC may be classified as one-session trauma group-specific information, whereby the primary posttraumatic physical information may be obtained based on a type of the traumatic event to which the individual is exposed.

Here, the heart rate indicates the number of heartbeats that increases or decreases based on a condition of the individual, and the HRV may be indicated using a low-frequency (LF)/high-frequency (HF) ratio measured from the individual. In addition, the SC may be indicated in proportion to a humidity of the skin based on a condition of the individual. In an example, in a case in which the individual feels anxiety in response to a traumatic event, the heart rate may sharply increase, the HRV may show an imbalance in sympathetic-parasympathetic nerves based on the LF/HF ratio, and the SC may increase.

Anxiety, denial, or threat which is a PTSD symptom to be presented by an individual in response to a traumatic event may cause a tension in a human body, which may lead to a physical disorder such as hyperventilation or hyperactivity. In particular, the heart is an internal organ that may first physically respond to hyperarousal of the body. Thus, the posttraumatic behavior problem predicting apparatus may measure the neurophysiological element that changes in response to an infiltration symptom, an avoidance symptom, a cognitive/emotional denial symptom, or an arousal/response symptom of the individual.

Referring to FIG. 2b, the posttraumatic behavior problem predicting apparatus may obtain the blood substance element flowing in blood vessels of the individual and to be used to determine a state of an oxidative nutrient. In detail, the posttraumatic behavior problem predicting apparatus may obtain the blood substance element to determine a symptom to be presented through the blood as a maladjustment symptom of the individual exposed to the traumatic event. The posttraumatic behavior problem predicting apparatus may collect a small amount of venous blood sample, and measure a concentration of a predetermined substance that reflects an immune function, oxidative stress, a neuroplasticity, or a hypothalamic-pituitary-adrenal (HPA) axis of the individual.

Here, the immune function may indicate a distribution concentration of immune cells included in the blood, the oxidative stress may indicate a distribution concentration of a blood coagulation substance included in the blood, and the neuroplasticity may indicate a presence and absence of blood to be supplied while a neural pathway of the brain is changed and reorganized structurally and functionally by external stimulation, experience, and learning. An element related to the HPA axis may indicate a ratio of a stress hormone secreted and released in the blood by stress that the individual may experience in response to exposure to trauma. In particular, a concentration of a substance that represents an inflammation-immune system in the blood may be classified as posttraumatic physical response information specific to a physical violence trauma group, and information related to a concentration of a female hormone-related substance in the blood may be classified as posttraumatic physical response information specific to a sexual violence trauma group.

In an example, in a case in which the individual feels anxiety in response to a traumatic event, an activity of an immune system of the individual may decrease, a distribution concentration of immune cells may decrease, and a blood supply ratio in the body may decrease as a concentration of a blood coagulation substance increases (oxidative stress) and a stress hormone is released in the blood (the HPA axis) due to stress.

Referring to FIG. 2c, the posttraumatic behavior problem predicting apparatus may obtain the neuroimaging element to determine a structure of a brain region highly related to violence or fear response. In detail, the posttraumatic behavior problem predicting apparatus may obtain the neuroimaging element as a neurological element to measure a connectivity or an activity of the brain region in response to a traumatic event.

The neuroimaging element may be a magnetic resonance imaging (MRI) image to be used to determine violence based on whether brain tissue is activated. The neuroimaging information may be relatively difficult to collect, and thus may be classified as the secondary posttraumatic physical information and utilized to predict a violent behavior.

In general, the amygdala and the hippocampus of the human brain are known as storing or erasing memories of a situation that a person experiences. In this example, memories about anxiety and fear caused by a traumatic event may be imprinted on the brain through a fear circuit including the amygdala and the hippocampus. After experiencing the traumatic event, anxiety and fear may be triggered by the imprinted memories of the traumatic event in a situation in which the person does not feel fear due to functional or structural characteristics of the fear circuit and a brain structure connected thereto.

In particular, to predict a posttraumatic violent behavior symptom, a responsiveness to a traumatic stimulus and the volume of each of the nucleus accumbens and the prefrontal cortex belonging to a brain reward circuit may be significant. That is, the nucleus accumbens may increase a sensitivity with respect to a violent behavior by inducing an emotion or a behavior to enhance the brain reward circuit, and the prefrontal cortex may act as a control tower that modulates the operation of the nucleus accumbens to restrain a presentation of the violent behavior.

That is, it may be predicted that the brain reward circuit may be activated and a violent behavior may be presented as the volume of the nucleus accumbens increases, the responsiveness of the nucleus accumbens with respect to the traumatic stimulus increases, the volume of the prefrontal cortex decreases, and the responsiveness of the prefrontal cortex with respect to the traumatic stimulus decreases.

Thus, the posttraumatic behavior problem predicting apparatus may obtain the neuroimaging element to determine whether the fear circuit and the reward circuit in the brain are activated, and whether a brain-nervous system is activated in response to the activation of the fear circuit and the reward circuit, thereby determining the connectivity, the activity, and the brain structure in response to the exposure of the individual to trauma.

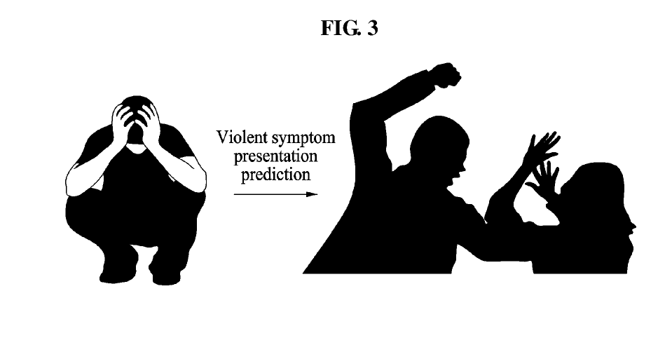

FIG. 3 illustrates an operation of predicting a presentation of a violent symptom according to an embodiment.

Referring to FIG. 3, a violent behavior problem predicting apparatus may analyze a neurophysiological element, a blood substance element, and a neuroimaging element as posttraumatic physical response information, and calculate a score for each element. Here, the score for each element may be calculated using a nonlinear equation between elements secondarily extracted from a sample group based on a type of a traumatic event, as described with reference to FIG. 1. Types of the extracted elements and the nonlinear equation may differ based on each type of the traumatic event. In view of the above, a posttraumatic behavior problem predicting apparatus may be individualized based on each type of the traumatic event, and a specific violent behavior problem predicting apparatus may be applied based on a type of the traumatic event to which an individual is exposed. That is, the posttraumatic behavior problem predicting apparatus may calculate the score for each element to determine a seriousness of a mental symptom of the individual exposed to the traumatic event based on each of the elements of the posttraumatic physical response information.

In this example, each element may have a different range of value indicating an anxiety symptom or a stress symptom of the individual exposed to the traumatic event based on a characteristic of each element, and thus the score calculated for each element may be normalized from “−1” to “1”. The posttraumatic behavior problem predicting apparatus may determine a characteristic of a future biological phenotype of the individual based on the normalized score of each element of the posttraumatic physical response information. In particular, to distinguish behavioral trauma to predict a violent behavior, a determiner of the posttraumatic behavior problem predicting apparatus may classify the individual as one of an emotional phenotype, a behavioral phenotype, and a cognitive phenotype through a cluster analysis based on the primary posttraumatic physical information. The posttraumatic behavior problem predicting apparatus may determine a problem behavior with a greatest value of a presentation index corresponding to a characteristic of the behavioral trauma to be a biological phenotype through the cluster analysis, thereby determining trauma of the individual.

The posttraumatic behavior problem predicting apparatus may extract a violent behavior risk group corresponding to the biological phenotype, and finally predict a violent behavior presentation probability by integrating the violent behavior risk group and the secondary posttraumatic physical information.

The posttraumatic behavior problem predicting apparatus may determine whether a future behavioral trauma group is generated immediately after exposure to trauma, using the nonlinear equation of the score of each element of the posttraumatic physical response information immediately after the exposure to trauma before a characteristic of the behavioral trauma group is presented. In an example, the posttraumatic behavior problem predicting apparatus may determine the characteristic of the biological phenotype which may be classified as future impulsivity, anger, attention, alcohol use, or the like based on the score of each of the neurophysiological element, the blood substance element, and the neurologic element.

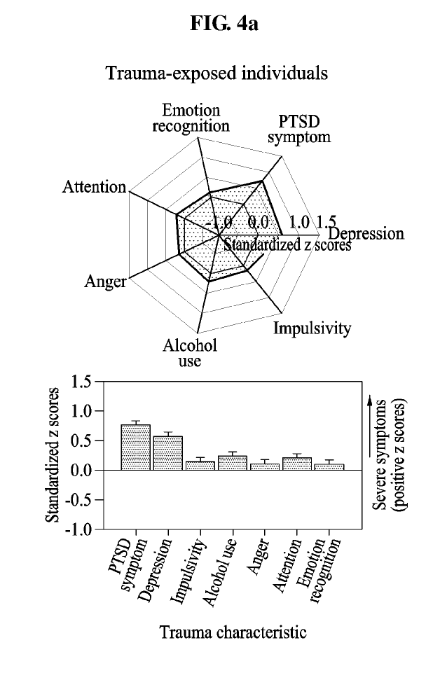

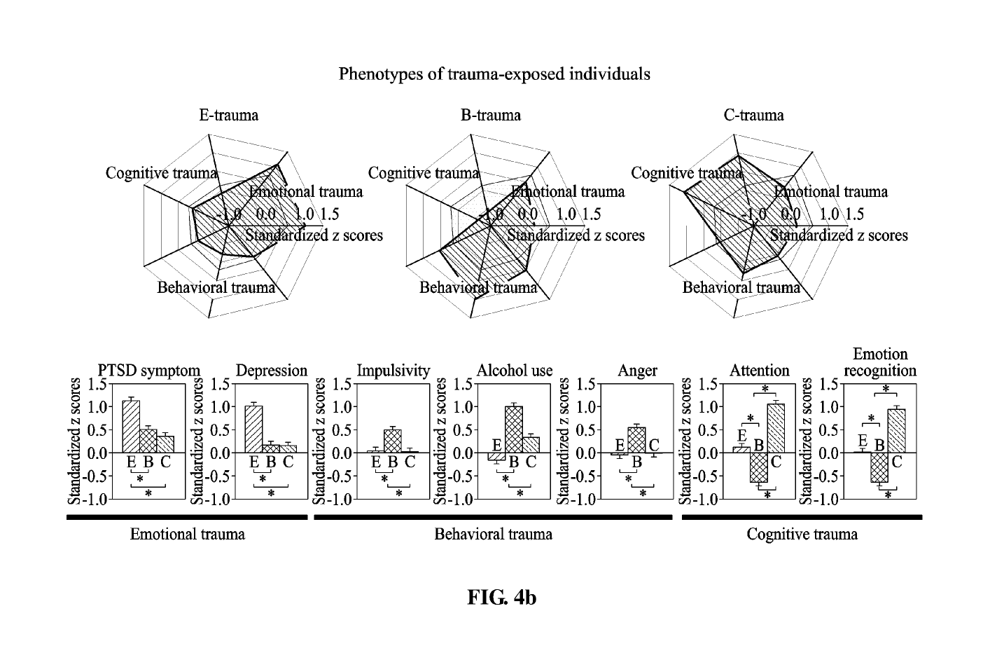

The posttraumatic behavior problem predicting apparatus may determine whether the biological phenotype is generated and a probability related thereto based on the characteristic of the biological phenotype to be generated in the future based on the posttraumatic physical response information obtained from the individual immediately after the exposure to trauma. In detail, the posttraumatic behavior problem predicting apparatus may analyze an odds ratio with which each element of the posttraumatic physical response information is classified as the biological phenotype. The posttraumatic behavior problem predicting apparatus may select a posttraumatic physical response factor with a high odds ratio for each biological phenotype. Characteristics of a behavioral trauma group to verify that a predicted cluster of the posttraumatic physical response factor is a cluster with a high probability of behavior problems are shown in FIGS. 4a and 4b.

Therefore, the posttraumatic behavior problem predicting apparatus may assume that a posttraumatic physical response factor with a great rate of predicting a high violent behavior risk group and with a low correlation between factors has a great power of explanation, and repeatedly analyze a combination of these various factors, thereby extracting factors that maximize the rate of predicting a high violent behavior risk group.

In detail, a predictor of the posttraumatic behavior problem predicting apparatus may extract the factors based on information related to a sample group for each type of the traumatic event, in an order of a factor having a highest index of correlation, by obtaining an index of correlation between each posttraumatic physical response factor (neurophysiological-neuroimaging response information) and trauma characteristics (outcome). Here, the trauma characteristics are information to be used to determine a biological phenotype group to be predicted herein, and may be expressed in an ascending order of presentation index scores specific to each characteristic.

In addition, the posttraumatic behavior problem predicting apparatus may apply a weight to a method of obtaining a posttraumatic physical response factor by incorporating an index of easiness therein if information is relatively easy to obtain. In an example, an index of easiness for obtaining a factor measurable by taking a small amount of venous blood sample may be calculated to be higher than that of a factor obtainable through a relatively complex neuroimaging analysis. As described with reference to FIGS. 2a through 2c, in a case in which factors have duplicate effects, the factors having duplicate effects may be excluded, and factors with a lowest correlation between factors and with a highest index of correlation with the trauma characteristics may be extracted to construct the nonlinear equation using a minimum number of factors. That is, the posttraumatic behavior problem predicting apparatus may primarily extract factors with high indices of correlation with the behavioral trauma and with high indices of easiness for obtaining information from the posttraumatic physical response factors. The posttraumatic behavior problem predicting apparatus may repeatedly analyze a combination with a lowest index of correlation between the primarily extracted factors, thereby finally selecting a nonlinear equation and a minimum number of posttraumatic physical response factors with a greatest predictability with respect to the behavioral trauma. The above process may be repeatedly performed based on each type of the traumatic event.

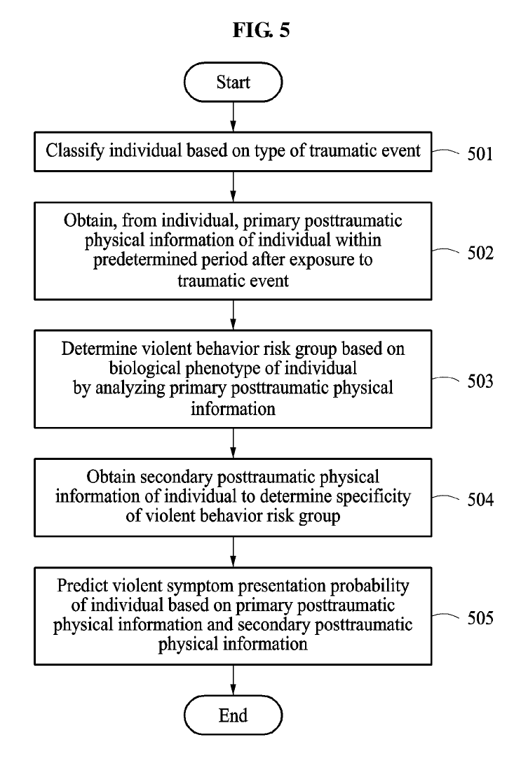

FIG. 5 is a flowchart illustrating a method of predicting a posttraumatic behavior problem according to an embodiment.

Referring to FIG. 5, in operation 501, a posttraumatic behavior problem predicting apparatus may classify an individual based on a type of a traumatic event to which the individual is exposed. The posttraumatic behavior problem predicting apparatus may classify the individual based on the type of the traumatic event that may be classified as physical violence, sexual violence, accidents/disasters, or others based on a characteristic of the traumatic event.

In operation 502, the posttraumatic behavior problem predicting apparatus may obtain primary posttraumatic physical information of the individual at an early stage within a predetermined period after the exposure the traumatic event. The posttraumatic behavior problem predicting apparatus may obtain a neurophysiological element and a blood substance element as the primary posttraumatic physical information of the individual. Here, the neurophysiological element may include biometric information related to autonomic nerve modulation of the individual. The blood substance element may include blood information related to oxidative stress, neuroregeneration and an HPA of the individual.

That is, the posttraumatic behavior problem predicting apparatus may obtain posttraumatic physical response information to be presented as a physically abnormal phenomenon to determine a presentation of a mental symptom and a posttraumatic symptom type with respect to the individual exposed to the traumatic event.

In operation 503, the posttraumatic behavior problem predicting apparatus may determine a violent behavior risk group based on a biological phenotype of the individual by analyzing the primary posttraumatic physical information of the individual. The posttraumatic behavior problem predicting apparatus may determine the biological phenotype based on a characteristic of the biological phenotype of the individual by applying a cluster analysis based on the posttraumatic physical response information. Here, the biological phenotype may have a characteristic of a biased phenotype of a predetermined symptom expressed by the individual for a short period of time or a long period of time as a mental maladjustment symptom after the individual is exposed to the traumatic event. That is, the characteristic of the biological phenotype may be used to verify a paraesthesia behavior presented by stress that the individual experiences. The posttraumatic behavior problem predicting apparatus may determine the biological phenotype of the individual through the paraesthesia behavior.

For this, the posttraumatic behavior problem predicting apparatus may calculate a score for each of the neurophysiological element and the blood substance element by analyzing the neurophysiological element and the blood substance element. The posttraumatic behavior problem predicting apparatus may calculate the score for each element to determine a seriousness with respect to the mental symptom of the individual exposed to the traumatic event based on each of the elements of the posttraumatic physical response information.

The posttraumatic behavior problem predicting apparatus may determine the characteristic of the biological phenotype of the individual based on the calculated score for each element. In an example, the posttraumatic behavior problem predicting apparatus may determine the characteristic of the biological phenotype which may be classified as impulsivity, anger, attention, alcohol use, or the like based on the score of each of the neurophysiological element, the blood substance element, and a neuroimaging element.

The posttraumatic behavior problem predicting apparatus may classify the individual as one of an emotional phenotype, a behavioral phenotype, and a cognitive phenotype by applying a cluster analysis based on the primary posttraumatic physical information. The posttraumatic behavior problem predicting apparatus may determine a biological phenotype corresponding to the classified phenotype to be a violent behavior risk group of the individual.

In operation 504, the posttraumatic behavior problem predicting apparatus may obtain secondary posttraumatic physical information of the individual to determine a specificity of the violent behavior risk group. The posttraumatic behavior problem predicting apparatus may obtain, as the secondary posttraumatic physical information, neuroimaging information about a brain reward circuit related to a violent behavior in brain tissue.

In operation 505, the posttraumatic behavior problem predicting apparatus may predict a violent symptom presentation probability of the individual based on the primary posttraumatic physical information and the secondary posttraumatic physical information. The posttraumatic behavior problem predicting apparatus may predict the violent symptom presentation probability in view of a correlation between the elements of the posttraumatic physical response information with respect to the biological phenotype. Here, the posttraumatic behavior problem predicting apparatus may predict the violent symptom presentation probability based on the primary posttraumatic physical information and the secondary posttraumatic physical information by applying a nonlinear equation extracted from a sample group classified based on the type of the traumatic event.

In an example, in a case of the classified type of the traumatic event to which the individual is exposed corresponds to 1) physical violence, 2) sexual violence, 3) accidents/disasters, or 4) others, the posttraumatic behavior problem predicting apparatus may predict the violent symptom presentation probability by applying a nonlinear equation extracted from 1) a physical violence sample group, 2) a sexual violence sample group, 3) a one-session trauma (accidents/disasters) sample group, or 4) other sample groups.

The posttraumatic behavior problem predicting apparatus may provide treatment criteria for a mental symptom of an individual predicted to have a high violent symptom presentation probability before a violent symptom is presented, thereby providing a basis for linking the individual to treatment before the mental symptom is aggravated.

Ultimately, different posttraumatic behavior problem predicting apparatuses corresponding to types of traumatic events to which a sample group is exposed may be utilized through machine learning of the sample group based on the types of the traumatic events. In addition, the posttraumatic behavior problem predicting apparatus may predict a characteristic of a posttraumatic violent behavior yet to be presented by the individual exposed to the traumatic event based on a posttraumatic physical response level collected immediately after the individual is exposed to the traumatic event.

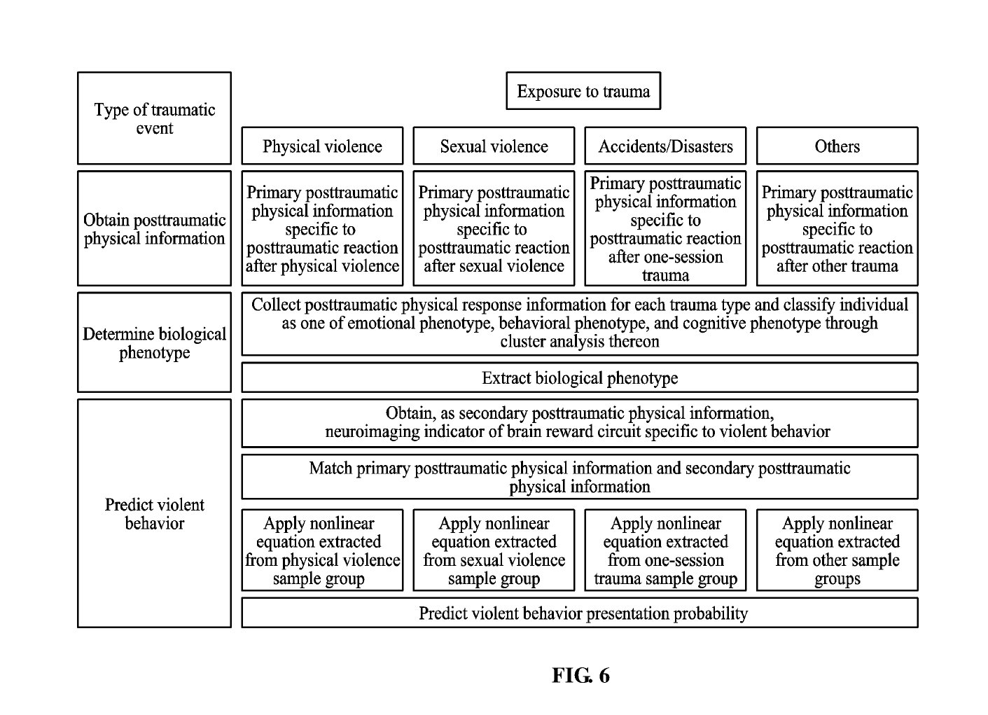

FIG. 6 is a block diagram illustrating an example of predicting a violent behavior problem of an individual according to an embodiment.

Referring to FIG. 6, a type of a traumatic event to which an individual is exposed may be classified as (i) physical violence, (ii) sexual violence, (iii) accidents/disasters, or (iv) others. After the exposure to the traumatic event, primary posttraumatic physical information specific to a posttraumatic response may be obtained from the individual. The primary posttraumatic physical information obtained based on the type of the traumatic event may be collected, and the individual may be classified as one of a behavioral phenotype, an emotional phenotype, and a cognitive phenotype through a cluster analysis thereon, and a biological phenotype may be extracted.

A violent behavior of the individual may be predicted based on the extracted biological phenotype. In detail, a neuroimaging indicator of a brain reward circuit specific to the violent behavior may be obtained as secondary posttraumatic physical information. Then, posttraumatic physical response information may be generated by matching the primary posttraumatic physical information and the secondary posttraumatic physical information.

A violent symptom presentation probability of the individual may be predicted by applying a nonlinear equation extracted from each traumatic event sample group based on the type of the traumatic event to the posttraumatic physical response information.

The various technologies described in this specification can be implemented as digital electronic circuitry, computer hardware, firmware, software, or combinations of these. The technologies can be implemented as a computer program, that is, an information carrier, for example, a computer program typically embodied in the form of a machine-readable storage (computer-readable medium) or a radio wave signal, to process operation of a data processing device, for example, a programmable processor, a computer, or a plurality of computers, or to control the operation. The computer program can be recorded in any form of program languages including a compiled language and an interpreted language, and can be developed in any form including an independent program or a module, a component, a subroutine, or any other unit suitable for use in a computing environment. The computer program may be deployed to be processed by one computer or multiple computers in one site, or distributed across multiple sites and interconnected through a communication network.

Processors suitable for the execution of a computer program include, for example, both general and special purpose microprocessors, and any one or more processors of any kind of digital computer. In general, a processor will receive instructions and data from a read-only memory (ROM), a random access memory (RAM), or both. Elements of a computer may include at least one processor for executing instructions and one or more memory devices for storing instructions and data. In general, a computer may include one or more mass storage devices, for storing data, for example, magnetic, magneto-optical disks, or optical disks, may receive data from them, may transmit data to them, or may be coupled to them to transceive data. Information carriers suitable for embodying computer program instructions and data include magnetic media, e.g., hard disks, floppy disks, and magnetic tapes, optical media, e.g., compact disk ROMs (CD-ROMs) and digital video disks (DVDs), magneto-optical media, e.g., floptical disks, semiconductor memory devices, e.g., RAMs, flash memories, erasable programmable ROMs (EPROMs), electrically erasable programmable ROMs (EEPROMs). The processors and the memories may be supplemented by or incorporated in special purpose logic circuitry.

Further, the computer-readable medium may be an arbitrarily available medium that may be accessed by a computer and may include a computer storage medium and a transmission medium.

While this specification includes details of a plurality of specific implementations, these should not be understood as limitations of any invention or the scope to be claimed, but should be understood as descriptions of features that can be peculiar to specific embodiments of the specific invention. Specific features described herein may be implemented by being combined in a single embodiment in the context of an individual embodiment. On the other hand, various features described in the context of a single embodiment may be implemented individually or in appropriate sub-combinations in a plurality of embodiments. While features may work in specific combinations and may be described as initially claimed so, at least one feature may be excluded from a claimed combination in some cases, and the claimed combination may be changed to a sub-combination or a modification of the sub-combination.

Similarly, although drawings illustrate operations in a particular order, this does not mean that these operations should be performed in the illustrated particular order or sequence or that all illustrated operations should be performed to obtain a desired result. In a particular case, multitasking and parallel processing may be advantageous. Separation of various system components in the above-described embodiments does not mean that such separation is required for all embodiments. In general, described program components and systems may be integrated in a single software product or may be packed in multiple software products.

Meanwhile, embodiments for exemplifying the technical spirit of the present invention have been described and shown above, but the present invention is not limited to shown and described configurations and effects. Those of ordinary skill in the art would appreciate that various changes and modifications of the present invention can be made without departing from the technical spirit. Therefore, it is to be understood that all suitable changes, modifications, and equivalents fall within the scope of the present invention.

1. A method of predicting a posttraumatic behavior problem, the method comprising:
classifying an individual based on a type of a traumatic event to which the individual is exposed;
obtaining, from the individual, primary posttraumatic physical information of the individual within a predetermined period after the exposure to the traumatic event;
determining a violent behavior risk group based on a biological phenotype of the individual by analyzing the primary posttraumatic physical information;
obtaining secondary posttraumatic physical information of the individual to determine a specificity of the violent behavior risk group; and
predicting a violent symptom presentation probability of the individual based on the primary posttraumatic physical information and the secondary posttraumatic physical information.

2. The method of claim 1, wherein the type of the traumatic event is classified as physical violence, sexual violence, accidents/disasters, or others based on a characteristic of the traumatic event.

3. The method of claim 1, wherein the primary posttraumatic physical information includes a neurophysiological element related to autonomic nerve modulation, and a blood substance element related to oxidative stress, neuroregeneration and a hypothalamic-pituitary-adrenal (HPA) axis of the individual.

4. The method of claim 1, wherein the determining comprises:
classifying the individual as one of an emotional phenotype, a behavioral phenotype, and a cognitive phenotype by applying a cluster analysis based on the primary posttraumatic physical information; and
determining a biological phenotype corresponding to the classified phenotype to be the violent behavior risk group of the individual.

5. The method of claim 4, wherein the biological phenotype has a characteristic of a biased phenotype of a predetermined symptom expressed by the individual for a short period of time or a long period of time as a mental maladjustment symptom after the individual is exposed to the traumatic event.

6. The method of claim 4, wherein the obtaining of the secondary posttraumatic physical information comprises obtaining, as the secondary posttraumatic physical information, neuroimaging information about a brain reward circuit related to a violent behavior in brain tissue.

7. The method of claim 1, wherein the predicting comprises predicting the violent symptom presentation probability of the individual in view of a correlation between elements based on the primary posttraumatic physical information and the secondary posttraumatic physical information.

8. An apparatus for predicting a posttraumatic behavior problem, the apparatus comprising:
a processor configured to:
classify an individual based on a type of a traumatic event to which the individual is exposed,
obtain, from the individual, primary posttraumatic physical information of the individual within a predetermined period after the exposure to the traumatic event,
determine a violent behavior risk group based on a biological phenotype of the individual by analyzing the primary posttraumatic physical information,
obtain secondary posttraumatic physical information of the individual to determine a specificity of the violent behavior risk group, and
predict a violent symptom presentation probability of the individual based on the primary posttraumatic physical information and the secondary posttraumatic physical information.

9. The apparatus of claim 8, wherein the type of the traumatic event is classified as physical violence, sexual violence, accidents/disasters, or others based on a characteristic of the traumatic event.

10. The apparatus of claim 9, wherein the primary posttraumatic physical information includes a neurophysiological element related to autonomic nerve modulation, and a blood substance element related to oxidative stress, neuroregeneration and a hypothalamic-pituitary-adrenal (HPA) axis of the individual.

11. The apparatus of claim 9, wherein the processor is configured to classify the individual as one of an emotional phenotype, a behavioral phenotype, and a cognitive phenotype by applying a cluster analysis based on the primary posttraumatic physical information, and determine a biological phenotype corresponding to the classified phenotype to be the violent behavior risk group of the individual.

12. The apparatus of claim 11, wherein the biological phenotype has a characteristic of a biased phenotype of a predetermined symptom expressed by the individual for a short period of time or a long period of time as a mental maladjustment symptom after the individual is exposed to the traumatic event.

13. The apparatus of claim 11, wherein the processor is configured to obtain, as the secondary posttraumatic physical information, neuroimaging information about a brain reward circuit related to a violent behavior in brain tissue.

14. The apparatus of claim 9, wherein the processor is configured to predict the violent symptom presentation probability of the individual in view of a correlation between elements based on the primary posttraumatic physical information and the secondary posttraumatic physical information.
